(19) United States
(12) Patent Application Publication
(10) Pub. No.: US 2012/0203093 A1
(43) Pub. Date: Aug. 9, 2012

(54) APPARATUS, SYSTEM AND METHODS FOR PHOTOACOUSTIC DETECTION OF DEEP VEIN THROMBOSIS

(76) Inventors: Mir Imran, Los Altos Hills, CA (US); Glen McLaughlin, San Carlos, CA (US)

(21) Appl. No.: 13/369,288

(22) Filed: Feb. 8, 2012

Related U.S. Application Data
(60) Provisional application No. 61/462,917, filed on Feb. 8, 2011.

(51) Int. Cl. A61B 6/00

(52) U.S. Cl. 600/407

(57) ABSTRACT

Embodiments of the invention provide apparatus, systems and methods for the detection of deep vein thrombosis (DVT) using photoacoustic measurement of hemoglobin in different states of oxgyenation within tissue. One embodiment of a system for DVT detection comprises at least a first and second light source that emit light at first and second wavelengths, an acoustic transducer, a data converter and a processor. The first and second light sources are directed on the patient's skin to produce a photoacoustic signal (PS) correlated to an amount of absorbance of the first and second wavelengths by a target region of tissue beneath the patient's skin. The acoustic transducer detects the PS and transduces it into an electrical signal which is correlated to the PS. The data converter converts the electrical signal into a digital signal which is analyzed by the processor to detect the presence of DVT within the target region.

### RELATED APPLICATIONS

This application claims the benefit of priority of Provisional U.S. Patent Application Ser. No. 61/462,917, entitled “APPARATUS, SYSTEM AND METHODS FOR PHOTOACOUSTIC DETECTION OF DEEP VEIN THROMBOSIS”, filed Feb. 8, 2011; which is fully incorporated by reference herein for all purposes.

### FIELD OF THE INVENTION

Embodiments described herein relate to a device, system and method for detection of deep vein thrombosis. More specifically, embodiments described herein relate to a device, system and method for detection of deep vein thrombosis using non-invasive photo acoustic detection methods.

### BACKGROUND

Thrombosis is the formation of a blood clot (also known as thrombus), inside a blood vessel. Thrombi (plural of thrombus) are capable of obstructing blood in a number of blood vessels in the body. In a relatively large vessel, the blood flow may simply be decreased whereas when the thrombus occurs in a relatively small vessel, blood flow may be significantly obstructed and in some cases, may result in the injury, deterioration and even death of the tissue supplied by the vessel.

When a thrombus occurs within the deep veins, those veins that are deep within the body, as opposed to superficial veins which are close to the surface of the skin, it is described as deep venous thrombosis or DVT. DVT typically affects the deep veins in the leg such as the femoral vein, popliteal vein, or the deep veins of the pelvis. Common symptoms of DVT include pain, swelling and redness of the affected area as well as dilation of the surface veins. While these conditions are not life threatening in themselves, the real risk of DVT occurs when a portion of thrombus breaks free and travels through the bloodstream to block a blood vessel in the lungs. Such a blockage, known as a pulmonary embolism, can cause sharp chest pain or breathlessness, and can be life-threatening if the circulating clot is large. The longer the clot causing a DVT is present, the greater the risk of pulmonary embolism. Moreover, in nearly half of all DVT cases, the patient has no symptoms so they are not even aware of the condition and the associated risk of pulmonary embolism. Not surprisingly, untreated lower extremity DVT has nearly a 3% mortality rate.

While the most common causes of DVT are recent surgery and hospitalization, there are several other known risk factors for DVT including age, obesity, infection, immobilization, contraception usage, tobacco usage, and air travel. These risk factors in turn affect one or more hemodynamic factors associated with the development of thrombus including: (1) rate of flow through the vessel, (2) the consistency or thickness of the blood flowing through the vessel, and (3) the quality of the vessel wall.

The development of DVT may be primary, also known as idiopathic, or secondary. In the case of idiopathic DVT, the development of DVT is unprovoked or unassociated with any known risk factor. The development of DVT may be considered secondary when associated with at least one known risk factor. It is estimated that 145 per 100,000 persons in the general population develop symptomatic DVT of which, 69 per 100,000 persons experience a pulmonary embolism. DVT disease remains a significant cause of mortality and morbidity despite widespread availability of effective prophylactic regimes in hospitalized patients.

DVT may be diagnosed through a variety of means including physical examination, imaging, and/or by performing blood tests for biomarkers associated with DVT. However, each has drawbacks including reliability, invasiveness, ease of use and cost. In a physical examination, DVT may be diagnosed by measuring the circumference of the affected contralateral limb at a particular point and palpating the venous tract. However, physical diagnoses are often unreliable for excluding a diagnosis of DVT.

Intravenous venography which is more reliable, involves injecting a peripheral vein of an affected limb with a contrast agent and taking X-rays to determine whether the venous supply has become obstructed. However, this approach is a very invasive procedure exposing the patient to risk of both X-ray and contrast agents in addition to the time and cost of the procedure. Ultrasound is another imaging technique, which although is less invasive, can be costly and requires the patient to travel to a hospital or medical center which is able to afford the equipment and personnel. Blood tests may also be utilized to test for biomarkers commonly associated with DVT, such as thrombin-antithrombin-complex (TAT) and fibrin/fibrinogen-degradation product (FDP) D-dimers. However, these tests are not always reliable and still involve the time and cost of performing the test with results not immediately available. This is particularly the case if outside laboratories are used (a common practice). Results may not be known for several hours or even days with the patient at risk for developing a pulmonary embolism all the while. Also, none of the current methods address the problem that DVT often develops when a person is immobilized for long periods such as during air travel. What is needed therefore, is an easier and more rapid method for detection of DVT's which can be performed in the doctor's (or other caregivers) office or by the patient at home or another location.

### BRIEF DESCRIPTION OF THE INVENTION

The conversion between light and acoustic waves due to absorption and localized thermal excitation is known as the photoacoustic effect. Various embodiments of the invention provide apparatus, systems and methods which utilize the photoacoustic effect to detect deep vein thrombosis (DVT) by detecting regions of tissue affected by the occlusion of a deep vein. The type of occlusions detected by embodiments of invention include those associated with deep vein thrombosis (detection of other types of occlusions is also contemplated, such as superficial vein occlusions and arterial occlusions). Such deep vein occlusions prevent the flow of oxygen rich blood to the effected region of tissue resulting in hypoxia including the presence of greater amounts of deoxygenated hemoglobin in relation to oxygenated hemoglobin. The ratio of oxyhemoglobin (i.e., oxygenated hemoglobin) to de-oxyhemoglobin provides a biomarker of DVT. Various embodiments of the invention provide novel approaches for detection of that ratio. For example, in one approach, embodiments of the invention provide a DVT detection system utilizing the photo acoustic effect (described below) to non-invasively measure the relationship between the concentrations of oxy-hemoglobin and deoxyhemglobin. That relationship is then utilized to provide information to the user for the detection of DVT in a patient.

Oxyhemoglobin and deoxyhemoglobin have frequency dependent light absorption properties, i.e., they absorb light differently at different frequencies of impinging light. Once absorbed, they transduce the light into sound waves by virtue of the photoacoustic effect with the resultant sound waves backscattered in tissue. Accordingly, the ratio of oxyhemoglobin to deoxyhemoglobin can be determined using both their transducer like properties and their frequency dependence light absorption properties. Embodiments of the invention can use these two properties to generate an acoustical-based map of the relative concentration intensities of each constituent. The metrics of these maps can then be utilized to extract the probability of an occlusion being present. In addition to an apparatus or device for the detection of DVT using ratios of oxyhemoglobin to deoxyhemoglobin, embodiments of the invention also contemplate an apparatus having an interchangeable photoacoustic transducer section. In use, such embodiments allow for measurement of a variety of organic or inorganic substances present within a target tissue region so as to be able to determine the presence of a variety of conditions, by allowing the user to change transducer sections so as to measure a particular organic and/or inorganic substance associated with a particular condition (e.g., diabetes as determined by measurement of glycosylated hemoglobin as a biomarker of hyperglycemia).

For purposes of explanation of how the relationship between oxygenated hemoglobin to deoxygenated hemoglobin can be obtained using the photoacoustic effect, a brief description will now be presented of the Beer Lambert law for the absorption of light. This law can be used to estimate and compensate for the decrease in light intensity as light travels through the medium under study. A simplified version of the basic governing equation for this law as it relates to oxyhemoglobin and deoxyhemoglobin is shown below.

I(r,λ)=I(λ)e^{-(α(λ)}^{ₒₓ}^{∫}^{ₚ}^{^{r}}^{c}^{ₒ}^{(l)dl+α(λ)}^{ᵣₓ}^{∫}^{ₒ}^{^{r}}^{c}^{ᵣ}^{(l)dl)r}

Where I is a channel based index, r is the distance from the light source to the point of photo acoustic transduction, λ is the frequency of the light source, αₒₓ is the absorption coefficient of HbO₂, αᵣₓ is the absorption coefficient of Hb, cₒ is the concentration of HbO₂ and cᵣ is the concentration of Hb.

$${Y}_{λ} ( r , θ ) = {\sum }_{i = 1}^{z} β ( i ) * ( {α}_{k} ( i ) {x}_{i} ( n - 1 ) + {γ}_{k} ( i ) {x}_{i} ( n ) + {ρ}_{k} ( i ) {x}_{i} ( n + 1 ) )$$

Where Y is the intensity of the photo acoustic reflection at a point r, θ; β is the aperture function, α, γ, ρ are the upsampling/interpolation coefficients x is the channel vector; n is the sample number of the channel vector. The sum is across all of the channel vectors.

$$S ( r , θ ) = \frac{{α}_{λ 1} {Y}_{λ 1} ( r , θ ) + … + {α}_{λ n} {Y}_{λ n} ( r , θ )}{{β}_{λ 1} {Y}_{λ 1} ( r , θ ) + … + {β}_{λ n} {Y}_{λ n} ( r , θ )}$$

Where S is the weighted average ratio at point r, θ of the photo acoustic intensity from each light frequency [symbol] ; [symbol] and [symbol] are the respective weighting coefficients of the photo acoustic image fields from each 1 to n light source and n is nth light source.

The overall signal to noise ratio (SNR) of these types of measurements has been traditionally fairly limited. In order to build up the SNR, data averaging can be used. This will enable the SNR to be improved by n*3 dB for every 2ⁿ increase in sampling. For example if you wanted to have 3 different light frequencies and improve the SNR by 6dB then a total of 12 light pulses/acoustic receive cycles would be required. Given that it takes a finite amount of time to collect these signals it is prudent to collect the Y_{λ} (r, θ) sets in a manner that minimizes errors due to motion (e.g., physical movement of the target tissue site from breathing, limb motion etc.). One approach for doing this would be to collect the photoacoustic image sequences as groups of frequencies of lights sources instead of just dwelling on a single frequency.

Once the respective image fields have been acquired and processed in the spatial domain, a figure of merit for the current state of the hemoglobin within the area of interest can be computed. Several methods can be used to compute the figure of merit. For example a spatial (r, θ) dependent weighting of the signals could be used based on their overall SNR and potentially a threshold level as well or something as simple as just a total sum of the direct signals or sum of the signal powers along with the appropriate threshold leveling as well if required. These signals could also be persisted over a time interval to further improve the overall SNR if desired.

One embodiment of a system for the detection of deep vein thrombosis in a patient comprises a first light source configured to emit light at a first wavelength, a second light source configured to emit light at a second wavelength, an acoustic transducer, a data converter and a processor. The first and second light sources are configured to be directed on the skin of the patient to produce a photoacoustic signal correlated to an amount of absorbance of the first and second wavelengths by a target region of the patient's tissue beneath the skin. In many embodiments, the first and second light sources are configured to emit substantially monochromatic light. A third light source may also be used, with that light source corresponding to a monochromatic source. One or more of the first, second or third sources may correspond to an LED, tunable LED, laser or tunable laser.

The acoustic transducer is configured to detect the photoacoustic signal and transduce that signal into an electrical output signal which is correlated to the photoacoustic signal. Put in another way, the acoustic transducer utilizes the photoacoustic signal to generate an output signal which is correlated to the photoacoustic signal. The acoustic transducer may comprise a piezoelectric crystal or other acoustic transducing material known in the art. The data converter converts the electrical signal into a digital signal and may correspond to an A/D converter. The processor is configured to analyze the digital signal to detect the presence of deep vein thrombosis within the target region. The processor may correspond to a microprocessor and may include one or more software modules, executable instruction sets or other logic for analyzing the digital signal.

Further details of these and other embodiments and aspects of the invention are described more fully below with reference to the attached figures.

### BRIEF DESCRIPTION OF THE DRAWINGS:

FIG. 1 is a block diagram of an embodiment of a photoacoustic-based DVT detection system.

FIG. 2 is a plot of the frequency dependent absorption characteristics of oxyhemoglobin and deoxyhemoglobin along with that of water.

FIG. 3 is a lateral view illustrating an embodiment of a transducer section for a photoacoustic-based DVT detection system

FIG. 4 is a schematic diagram of the electronic components used in an embodiment of the photoacoustic sensor element.

FIG. 5 illustrates an embodiment of a photoacoustic-based DVT detection apparatus including a user control panel along with a display, a handle and a transducer device.

FIG. 6 illustrates an embodiment of a photoacoustic-based DVT detection apparatus having interchangeable transducer modules.

### DETAILED DESCRIPTION OF THE INVENTION:

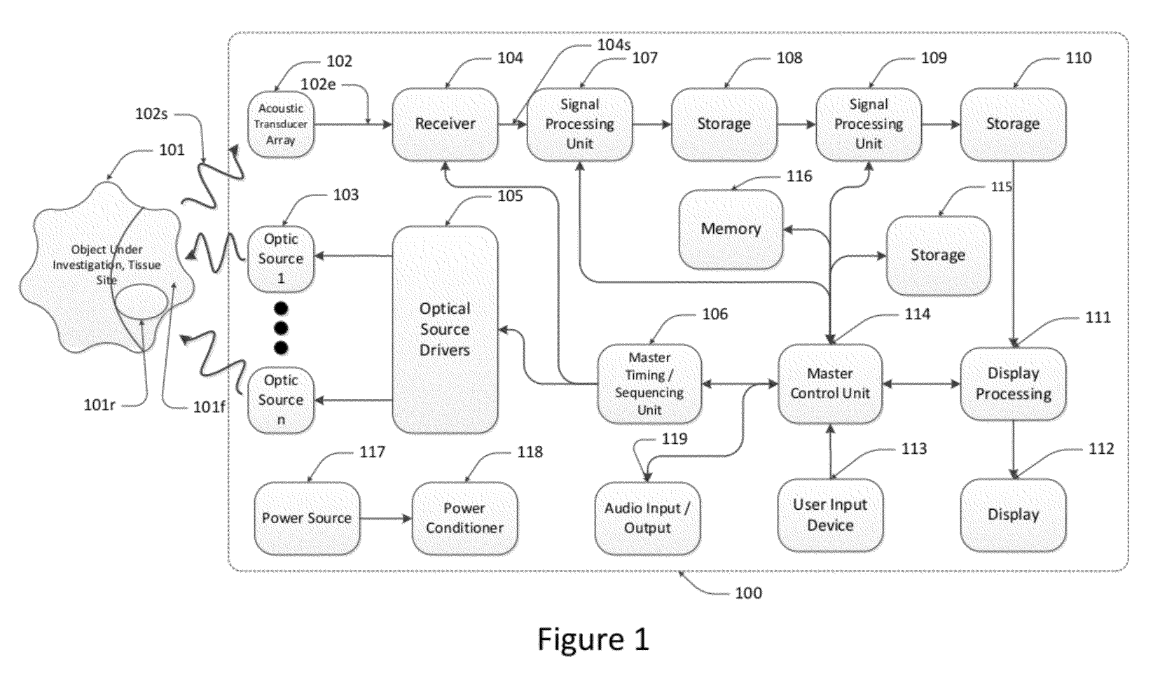

Embodiments of the invention provide apparatus, systems and methods for the detection of deep vein thrombosis (DVT) and/or other tissue conditions based on the detection of hemoglobin or other biomarkers. Referring now to FIG. 1, an embodiment of a system 100 for detection of a DVT or other condition in a region of tissue 101r (also described herein as tissue region 101r) in a human or other object under investigation 101 is depicted. Object 101 can be a target tissue site of a human or animal, a separate tissue sample, or even a solid or liquid. For ease of discussion, object 101 will now be referred to as a tissue site 101 (which may be located in/on a human or other animal) also referred to herein as a target tissue site 101, but it should be appreciated, that other forms of object 101 are equally applicable. Also, the tissue region 101r at tissue site 101 can encompass one or both of the skin and underlying tissue, though in many applications, it will comprise a region (which can be volume or area) of tissue beneath the skin. For embodiments of the invention configured for detection of DVT, tissue site 101 will often be the leg of the patient but may also be the arm, torso or neck or other area of the body. Besides DVT, other tissue conditions which can be detected by system 100 include other types of occlusions such as arterial occlusions and occlusion of non-deep veins than those typically occluded by DVT. Still other conditions which can be detected by embodiments of system 100 include one or more of tissue ischemia and/or hypoxia due to other factors besides vessel occlusion (e.g., low hematocrit and/or low blood oxygen saturation).

System 100 is configured to interact with tissue at selected tissue site 101 so as to detect a DVT within a target region 101r at tissue site 101. According to one or more embodiments, this can be accomplished by means of one or more light sources 103 that are configured to illuminate tissue at the tissue site 101 so as to generate an optical illumination field 101f. Optical illumination field 101f has specific frequency characteristics that are based in part on the frequency and other properties of the light source 103. The frequency characteristics of the optical illumination field 101f are selected so as to have the field produce a photoacoustic output signal 102s (herein photoacoustic signal 102s) that is used in the detection of DVT's. This is due to the fact that the photoacoustic signal 102s depends upon the molecular composition of tissue within tissue site 101 for example, the amount of oxy vs. deoxyhemoglobin present within the tissue site. Light sources 103 may correspond to an array or family of light sources with each source having a different or the same frequency of light. In many embodiments, light sources 103 comprise at least a first and a second light source, with a third light source also contemplated. In one or more embodiments, light sources 103 may correspond to an LED, frequency tuned LED, laser, or other light emitting device known in the art. At least one of the first, second or third light sources comprise a substantially monochromatic light source such as an LED (light emitting diode) or laser.

The power and pulse profile required to drive the optic source 103 is provided by the optical source drivers 105 which may correspond to one or more analog power devices known in the art (e.g., a power amplifier). Control of the optical source driver 105 is provided by a master timing/sequencing unit 106, which may correspond to a microprocessor or an analog-based logic device. The master timing/sequencing unit 106 may be configured to handle all or a portion of the timing critical control of operations performed by system 100. For example, one such operation which may be controlled by unit 106 may include control of the time when the light sources 103 are activated to the control of the reception by the receiver 104 of the photoacoustic signal 102s.

A discussion will now be presented on the generation of a photo acoustic signal 102s using light source 103. As discussed above when lights sources 103 emit light onto the patient's skin at target tissue site 101, they create an illumination field 101f. This field is absorbed and scattered as it passes through the tissue comprising the target region 101r at tissue site 101. As the illumination field 101f is absorbed by tissue, a sound wave 102s is created which is also described herein as photoacoustic signal 102s. The sound wave 102s then travels through and out of tissue at tissue site 101 and is detected/received by the acoustic transducer array 102. The acoustic transducer array 102 converts the received sound wave (i.e., the photo acoustic signal 102s, also described as photoacoustic sound wave 102s) from the acoustic domain into a signal 102e in the electrical domain which is an electrical representation of photoacoustic sound wave 102s. This electrical signal is then routed via a wire to a receiver element 104 (herein receiver 104). The receiver 104 can be configured to have the ability to buffer and/or amplify the electrical signal generated by transducer array 102. It may correspond to an amplifier or an amplifier including an analog to analog to digital converter. The receiver 104 can also be configured to compensate for the predicted attenuation that the photoacoustic sound wave 102s would have experienced as it traveled through the tissue site, 101, by a time dependent gain. This time dependent gain can be synchronized to the illumination of the optic source, 103. In one or more embodiments, the timing control on how the amplifier parameters should vary based on the optic source 103, transmit can be controlled via a master timing/sequencing unit 106 which may correspond to a field programmable gate array (FPGA). After electrical signal 102e has been conditioned by receiver 104, it is then transferred to a signal processing unit 107 which may correspond to a digital signal processor. The signal processing unit 107 can be configured to filter or otherwise manipulate the incoming signal 104s from receiver 104. In one embodiment, manipulation can comprise mixing, for example, with a digital based mixer. In other embodiments, the manipulation can comprise filtering of acoustic noise due to motion of object 101 and/or ambient acoustic noise surrounding the patient. In particular embodiments processing unit 107 can be configured to filter out noise present in the environment on an airplane (jet or propeller) so that system 100 can be used detect DVT's in flight by a patient user. The signal processing unit 107 can also be configured to be able to average the data from the receiver 104 for example, on a transmit by transmit basis (other averaging methods known in the signal processing arts also contemplated).

After the signal processing unit 107 has completed processing of the data it is transferred to a storage unit/device, 108 (also referred to herein as storage 108). The data stored in storage 108 may comprise a matrix of signals xᵢ(n) also described herein as a photoacoustic dataset (where x is the magnitude of the electrical signal of channel I and n is the index of the time based sample). In a preferred embodiment, storage of the data is done based on the sample xᵢ(n). After the data from a photoacoustic data set has been stored in the storage 108, it is then transferred to a second signal processing unit 109 for image formation. Signal processor 109 may also correspond to a digital signal processor. In some embodiments, the image formation can be part of the initial signal processing unit, 107 so that it need not be done by processing unit 109. However, in preferred embodiments, it is done by processing unit 109. Moreover in such embodiments, storage 108 is desirably placed between the two signal processing units, 107 and 109, so as to buffer the data so that the processing performance of the signal processing unit, 109, can be spread out over the optic source pulse rate instead of the data reception rate (however, it will be appreciated that storage 108 need not be so configured and other placements and/or functions of storage 108 are contemplated). The image formed data is now placed in a storage or memory device, 110 (also referred to herein storage 110). In one or more embodiments, one or more of storage devices 110, 108 and other storage devices described herein may correspond to, RAM, DRAM, ROM, EPROM and other memory resources known in the art. Also one or more of processing unit 107, 109 and master control unit 114 and other processing units described herein, may correspond to a microprocessor, state device, ASIC (application integrated circuit), programmable logic controller, analog-based logic device or other logic device or resources known in the art. Also one or more of units 107, 109 and 114 or other processing unit described herein may include a software module or other executable instruction set for performing one or more processing steps.

In various embodiments, the master control unit, 114 can be configured to configure the two signal processing units, 107 and 109, so that they can provide optimum processing based on the characteristics of optical source 103 along with those of the acoustic transducer array, 102. The reconstructed data from storage, 110, is passed to the display processing, 111, where the data can be conditioned for the desired display properties. The data from storage, 110, can consist of a number of data sets generated by repeated pulse/receive cycles of 103 and 102, or it can contain just a single set. This is determined based on the parameters being extracted from the data. For example, for measuring the total oxyhemoglobin and deoxyhemoglobin within the tissue site, 101, typically 2 to 3 data sets are used at known frequencies of optical absorbance. These data sets can be manipulated either coherently or non-coherently for example, after magnitude detection. If it is desired to display to total % SpO₂ than after manipulating the image data the resulting sets can be summed together to get a single figure of merit. If however, it is desired to display an image set than the data from the image sets can be added across data sets on a spatial point by point basis and converted to display the desired image data on the display, 112. A master control unit, 114, coordinates the responses of the user to the user input device, 113, as well as coordinates the configuration of the master timing/sequencing unit 106. In various embodiments, control unit 114 may correspond to a processor such as microprocessor, a state device or an analog-based logic device. It may include logic, such as software module or other executable instruction set, for performing various data transformations or other operations. The master control unit 114 also desirably has a storage device 116, herein storage 116. Storage 116 which may correspond to a memory device 116 such as a RAM, DRAM, SRAM, DDR) which may be integral to or otherwise operably coupled to or associated with control unit 114. Storage 116 desirably has memory capabilities sufficient for keeping a history of user inputs or previous values. The storage 116, may also be associated with the master control unit 114, and contains the non-volatile information required to run the system 100 as well as a history of the past measurements. In addition to one or more of the proceeding components an audio input/output device 119 is also connected to the master control unit 114 and in one embodiment may correspond to a COEDEC. The audio input/output device 119 can sound an alarm if a measurement (e.g., an amount of oxyhemoglobin) is below a threshold or could take commands from the user by a voice recognition method. Power for device 119 is provided from an electrical power source, 117, which may correspond to a portable battery such as a lithium ion battery (or other electrical energy storage device) or an AC power source (e.g. provided by connection to a wall outlet). The power source 117 transfers power to a power conditioner device 118, such as a DC to DC converter or an AC to DC converter. The power conditioner device 118, unit then transfers power to the other pertinent devices and components of system 100. Power source 117 may also be supplied with power from a power storage device (not shown) such a portable battery or a super capacitor.

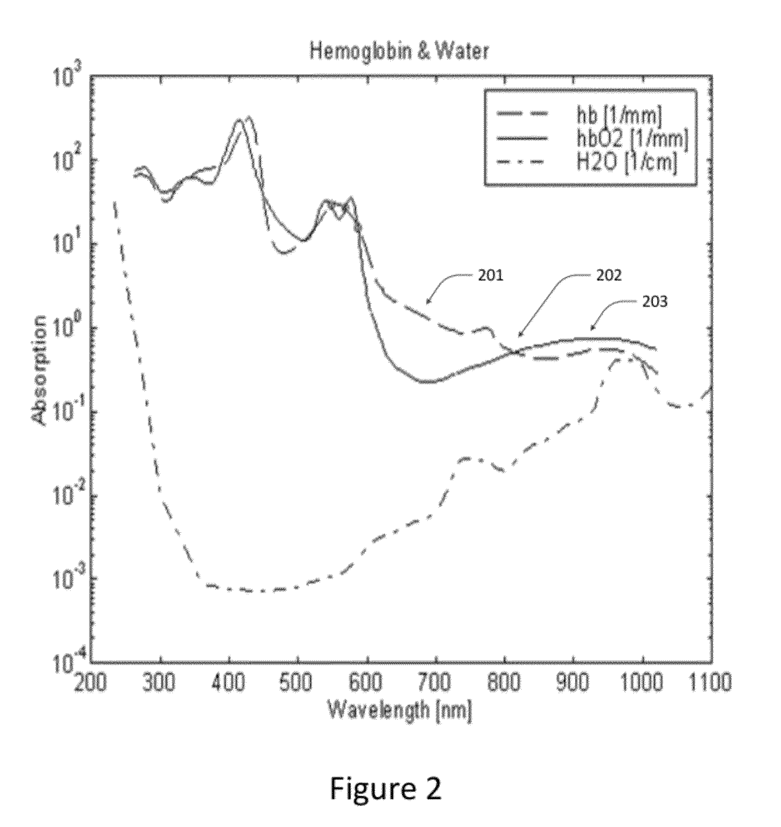

Referring now to FIG. 2, a plot of the absorption characteristics of oxyhemoglobin, deoxyhemoglobin and water is shown at different frequencies of illumination. In this figure, item 201 represents frequency of illumination where the absorption characteristics of deoxyhemoglobin are higher than oxyhemoglobin. While item 202 represents a frequency of illumination at which the absorption characteristics of deoxyhemoglobin are equal to those of oxyhemoglobin are equal, and item 203 represents a frequency of illumination at which the absorption characteristics of oxyhemoglobin are higher than that of deoxyhemoglobin. By using these different illuminating frequencies, a profile of the ratio of the relative constituents concentrations of oxyhemoglobin and deoxyhemoglobin can be calculated so that the necessary parameters can be displayed. In various embodiments, points on the plot shown in FIG. 2 in addition to those at 201, 202, and 203 may be used for calculation of desired parameters. Alternatively, fewer points can be used where, for example, just points 201 and 203 are used for the accurate calculation of the desired parameters.

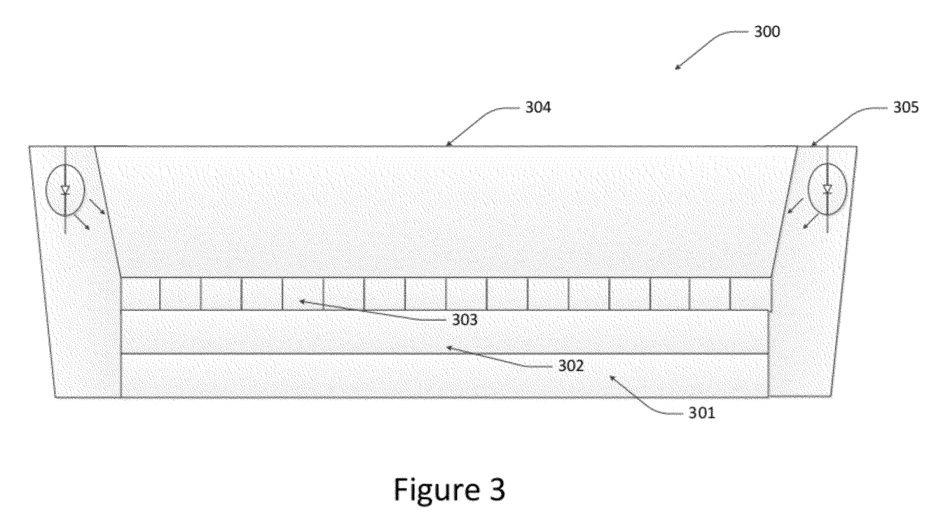

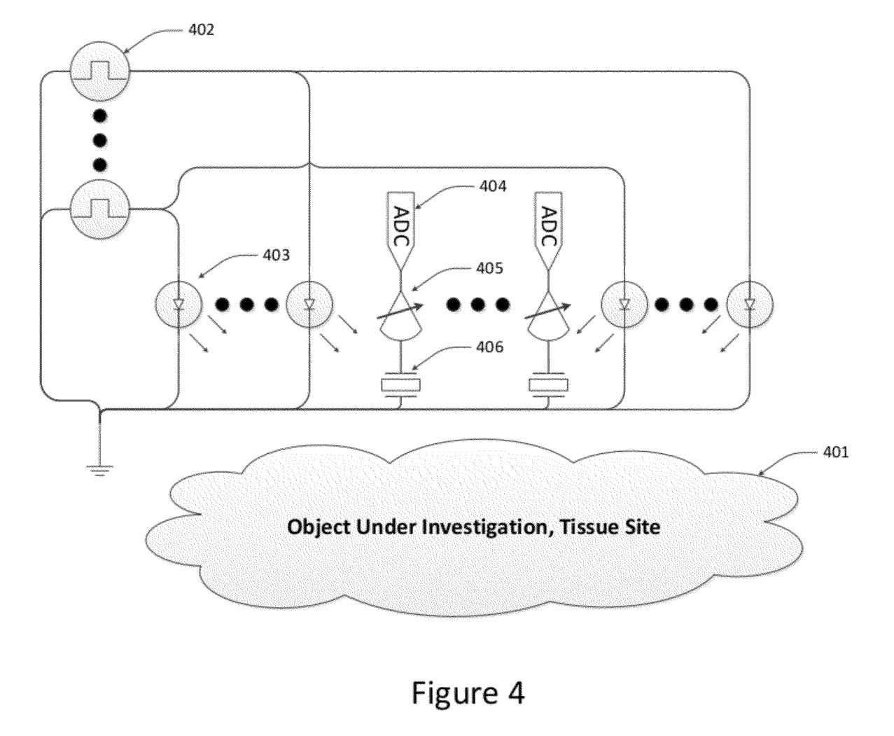

Referring now to FIG. 3, a depiction of an embodiment of a photoacoustic transducer 300 is shown. The transducer 300 can include one or more of an acoustic lens 301, matching layer 303, backing block 304 and light guide 305. Acoustic lens 301 may be fabricated from optically clear silicone rubber and is configured to concentrate and/or focus acoustical energy received from the tissue site and can also be configured to function as a patient isolation barrier. Also a matching layer 302 may be used to match the acoustic impedance of the tissue and the lens/patient isolation barrier to that of the transducer crystal elements, 303. Multiple matching layers 302 can be used to improve the overall transfer efficiency of the acoustic energy. A backing block 304 is used to absorb and disperse any acoustic energy that passes through the transducer crystal elements 303 so reflections can be minimized. According to the embodiment shown in FIG. 3, a light guide 305 may be placed around the periphery of the transducer 300 so as to transfer optical energy from a light source 403 (shown in FIG. 4 but shown in this FIG. 3) to the tissue site 101. Light guide 305 may correspond to an optical fiber or other light guide known in the art.

Various embodiments of the invention contemplate different methods and configurations for coupling the light source 403 and the transducer crystal elements 303. For example, in one or more embodiments, the light source 403 can be positioned in-line with the crystal elements 303n by the use of a transparent acoustic reflector where the light illuminating the tissue site is now collinear with the reflections of the acoustic signals generated from the light. In an additional or alternative embodiment, fiber optic cables can be used to conduct light from the light source 403 where the cable can be built directly into the transducer 300.

Referring now to FIG. 4, an embodiment of an apparatus 400 for generating and receiving the photo acoustic signals from a selected tissue site 401 is depicted. The apparatus may include a light source 403, a signal generator 402, acoustic transducer 406, amplifier 405, and data converter 404 (e.g., an analog to digital converter). In many embodiments, signal generator 402 may correspond to a pulse generator, 402, which can be configured to generate an electrical signal to drive the light source. The pulse generator, 402, is electrically connected to light source 403 (also described as light conversion device 403) which converts electrical signals to optical energy, 403. Any number of light sources can be employed. For example in one embodiment, light source 403 may correspond to a set of frequency tuned LEDs having quantum dot filters. In another embodiment, it may correspond to a tunable laser. Where fixed frequency light sources are used, than a family of light sources cane be used one at each desired frequency. The light source 403 transmits light energy 403′ into the tissue site, 401, which then selectively absorbs the light based on the frequency dependent properties of the molecular composition (e.g., oxyhemoglobin, deoxyhemoglobin) within the tissue site, 401. The absorbed energy is then converted from light energy into acoustic energy in the form of an acoustic wave or signal 407 (also described herein as photo acoustic signal 102s. Acoustic signal 407 travels from the point of origin 401′ in the tissue site, 401, to transducer 406 for conversion into electrical energy or signal 406′. Signal 406′ may be amplified by an amplifier, 405 which may correspond to an operational amplifier or op-amp 405. In one embodiment, amplification may be time gated or otherwise time dependent based on the time difference from when the light energy source 403 emits signal 403′ and acoustic transducer, 406 receives acoustic signal 407. Signals 406′ are than digitized by an A/D converter or other data conversion devise 404, into digital signal 404′ so that they can be further processed in the digital domain for one or more of analysis, image formation and DVT or other clinical condition prediction.

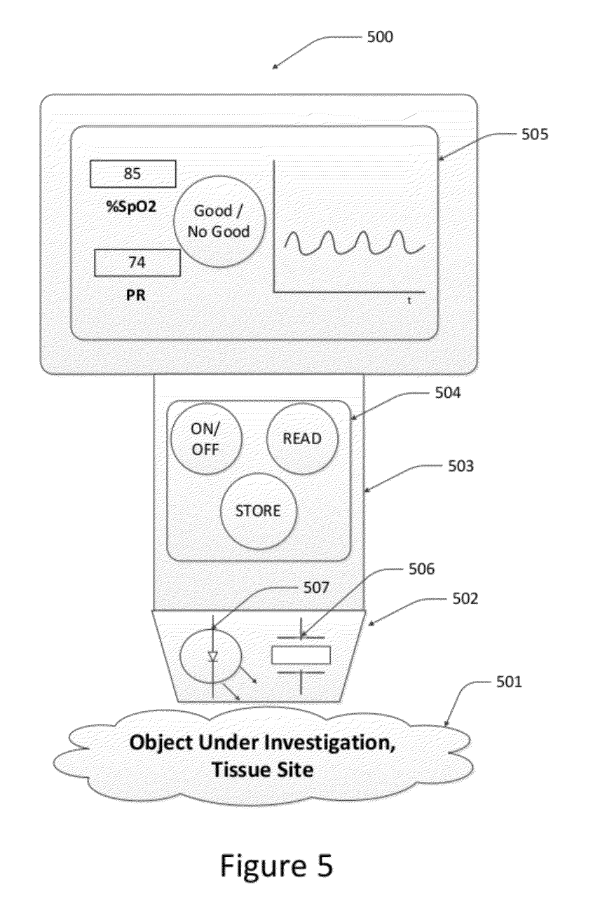

Referring now to FIG. 5, an embodiment of a DVT detection apparatus 500 (also referred to as unit 500) is depicted. The apparatus 500 includes a transducer module 502 having a light source 507 for the generation and transmission of optical signals to the tissue site or other object under investigation 501 and a transducer 506 for the reception of acoustic signals from the tissue site. Module 502 is attached to the handle of the unit, 503. The unit's handle, 503, is designed to be easily grasped by a single hand while allowing the user to simultaneously access a set of user controls, 504, with the same hand or the opposite hand. These user controls 504, are used to manipulate the actions of the apparatus for example to take a reading or measurement, store data from a reading and perform other related functions (e.g., wirelessly signal data, turn the apparatus on or off, view a map of the measured data or an image of the area to be analyzed for DVT's). The information is displayed on the user interface, 505, the user interface can be implemented in a number of ways from a LCD panel for a system that would display multiple sets of information to just a single light for a system with only a binary output. It would not be a limitation of this invention to integrate both the user input, 504, into a part of the system display, 505, as part of a touch panel.

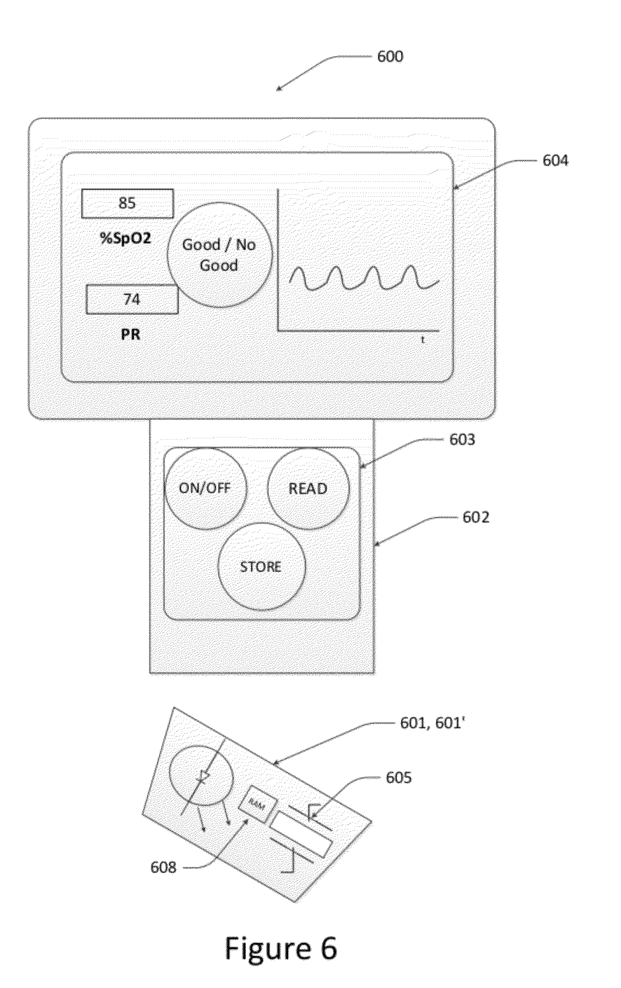

Referring now to FIG. 6, an embodiment of a DVT detection apparatus 600 having interchangeable transducer modules 601, is depicted. In this and related embodiments, apparatus 600 can be configured to allow the transducer module, 601 to be removed from the system handle 602 and be replaced with another module 601′. The other module 601′ can be used to detect one more organic or inorganic compounds besides hemoglobin. In one or more embodiments, transducer modules 601 can include a memory device 608 such as RAM, DRAM, ROM, EPROM, etc. or other memory resource known in the art so that i) all or a portion of the pertinent system parameters required to configure the apparatus 600 can be stored within the transducer module, 601; and ii) the apparatus can automatically configure itself for operation without the need for user input or other intervention.

### CONCLUSION

The foregoing description of various embodiments of the invention has been presented for purposes of illustration and description. It is not intended to limit the invention to the precise forms disclosed. Many modifications, variations and refinements will be apparent to practitioners skilled in the art. In particular embodiments, various modifications of system 100 can be made to make the system flight worthy so as allow a patient user to use in flight. Such modifications can include one or more of electrical, acoustic and acoustic shielding to prevent unwanted noises sources from interfering with operation of the system including optical and acoustic aspects of the operation of the system. The system may also be modified to detect a precurser state of a DVT state and then alert the patient to take appropriate action such as getting and walking around as to increase circulation in the target tissue site such as the leg. Such precursor states can include low levels of oxy-hemoglobin and/or high levels of deoxyhemoglobin.

Also, while embodiments of the invention are useful for detection of the state of hemoglobin within a region of interest (e.g., a volume of tissue in the leg) in a human or other animal, embodiments of the invention can also be used detect in a human or other animal a number of other compounds both organic and inorganic. For example, embodiments of the invention can also be used to detect glycosylated hemoglobin for long term measurement of blood glucose levels. Embodiments of invention can also be used to detect in vivo various biomarkers of a number of diseases and conditions and then use that information to make diagnostic predictions about the presence of the disease or condition. Such biomarkers and associated diseases and conditions can include cancer (e.g., PSA, PAP, tPSA, fPSA, proPSA, PSAD, PSAV, PSADT, EPCA, and EPCA-2, for prostate cancer), diabetes (low levels of insulin), heart attack (cardiac markers such as troponin, creatine kinase, Glycogen phosphorylase isoenzyme BB etc. for heart attack) and Alzheimer's (beta amyloid). Still other biomarkers of other these and other conditions are also considered.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Hence, the scope of the present invention is not limited to the specifics of the described embodiments, but is instead limited solely by the appended claims.

What is claimed is:

1. A system for detection of deep vein thrombosis in a patient, the system comprising:
a first light source configured to emit light at a first wavelength;
a second light source configured to emit light at a second wavelength, wherein the first and second light sources are configured to be directed on the skin of the patient to produce a photo acoustic signal which is correlated to an amount of absorbance of the first and second wavelengths by a target region of the patient's tissue;
an acoustic transducer for detecting the photo acoustic signal; wherein the acoustic transducer generates an electrical signal resulting from the photo acoustic signal;
a data converter for converting the electrical signal into a digital signal; and
a processor configured to analyze the digital signal to detect the presence of deep vein thrombosis within the target region.

2. The system of claim 1, wherein the target region is beneath the patient's skin.

3. The system of claim 1, wherein the first wavelength is preferentially absorbed by oxy-hemoglobin and the second wavelength is preferentially absorbed by deoxy-hemoglobin.

4. The system of claim 1, wherein at least one of the first or second light sources comprises a substantially monochromatic light source.

5. The system of claim 1, further comprising a third light source configured to emit at a third wavelength; wherein the first, second and third light sources are configured to be directed on the skin of the patient to produce a photo acoustic signal which is correlated to an amount of absorbance of the first, second and third wavelengths by the patient's tissue in the target region.

6. The system of claim 1, wherein at least one of the first or second light sources comprises a laser.

7. The system of claim 1, wherein at least one of the first or second light sources comprises at least one LED device.

8. The system of claim 7, wherein the at least one LED device comprises a frequency tuned LED device.

9. The system of claim 7, wherein the at least one LED device comprises at least one quantum dot filter for improving spectral purity of light emitted by the LED device.

10. The system of claim 1, wherein the processor includes logic for performing data averaging to improve signal to noise ratio (SNR).

11. The system of claim 10, wherein the processor includes logic for performing data frame averaging to improve SNR.

12. The system of claim 1, wherein the processor includes logic for analyzing a figure of merit to determine an oxygenated state of hemoglobin within the target region of tissue.

13. The system of claim 12, wherein the figure of merit is a time dependent figure of merit.

14. The system of claim 1, wherein the processor includes logic for determining a hemodynamic parameter or pulse rate of the patient.

15. The system of claim 1, wherein the processor includes logic for generating a region image display based on an intensity of an oxygenated state of hemoglobin within the target region.

16. The system of claim 1, wherein the processor includes logic for generating a region image display based on a signal power of an oxygenated state of hemoglobin within the target region.

17. The system of claim 1, wherein at least one of the first or second light sources is configured to generate a photo acoustic signal which is correlated to an amount of material other than hemoglobin (non-hemoglobin material) present in the patient's tissue.

18. The system of claim 17, wherein the non-hemoglobin material present in the patient's tissue comprises organic or inorganic material.

19. The system of claim 1, further comprising:
memory resources associated with at least one of the processor or the transducer, the memory resources configured to store system parameters relating to the acoustic transducer.

20. The system of claim 1, further comprising:
an audio alarm operably coupled to the processor, the audio alarm configured to generate an audio alarm when a threshold level of hemoglobin has been detected in the tissue region.

21. The system of claim 20, wherein the hemoglobin is hemoglobin in an-oxygenated state.

22. The system of claim 21, wherein the threshold level is a minimum level of hemoglobin in an oxygenated state.

23. The system of claim 20, wherein the threshold level of hemoglobin is a ratio of an amount of hemoglobin in a de-oxygenated state to an amount of hemoglobin in an oxygenated state.

24. A system for detection of deep vein thrombosis in a patient, the system comprising:
a first light source configured to emit substantially monochromatic light at a first wavelength;
a second light source configured to emit substantially monochromatic light at a second wavelength, wherein the first and second light sources are configured to be directed on the skin of the patient to produce a photo acoustic signal which is correlated to an amount of absorbance of the first and second wavelengths by a target region of the patient's tissue beneath the skin;
an acoustic transducer for detecting the photo acoustic signal; wherein the acoustic transducer generates an electrical signal resulting from the photo acoustic signal;
a data converter for converting the electrical signal into a digital signal; and
a processor configured to analyze the digital signal to detect the presence of deep vein thrombosis within the target region.

25. A system for detection of a tissue condition in a patient, the system comprising:
a first light source configured to emit light at a first wavelength;
a second light source configured to emit light at a second wavelength, wherein the first and second light sources are configured to be directed on the skin of the patient to produce a photo acoustic signal which is correlated to an amount of absorbance of the first and second wavelengths by a target region of the patient's tissue;
an acoustic transducer for detecting the photo acoustic signal; wherein the acoustic transducer generates an electrical signal resulting from the photo acoustic signal;
a data converter for converting the electrical signal into a digital signal; and
a processor configured to analyze the digital signal to detect the presence of the tissue condition within the target region.

26. The system of claim 25, wherein the condition is deep vein thrombosis and the first and second wavelengths are selected to be preferentially absorbed by oxy-hemoglobin and deoxy-hemoglobin.

27. The system of claim 25, wherein the condition is hypoxia.

28. The system of claim 25, wherein the condition is a blood glucose level.

29. The system of claim 28 wherein one of the first or second wavelengths is selected to be preferentially absorbed by glycosolated hemoglobin.

30. The system of claim 25, wherein the condition is a condition associated with cancer.

31. The system of claim 25, wherein the condition is a level of insulin.

32. The system of claim 25, wherein the condition is a heart attack.

33. The system of claim 32, wherein at least one of the first or second wavelengths is selected to be preferentially absorbed by a biomarker of a heart attack.

34. The system of claim 33, wherein the biomarker is at least one of troponin, creatine kinase, glycogen phosphorylase isoenzyme BB.

35. A method for detection of deep vein thrombosis in a patient, the method comprising:
emitting light at a first and second wavelength onto the skin of a patient at a target tissue region,
generating a photo acoustic signal in the target region, the photo acoustic signal being correlated to an amount of absorbance of the first and second wavelengths;
transducing the photo acoustic signal into an electrical signal; and
analyzing the electrical signal to detect the presence of deep vein thrombosis within the target region.

36. The method of claim 35, wherein the target region is beneath the patient's skin.

37. The method of claim 35, wherein the first wavelength is preferentially absorbed by oxy-hemoglobin and the second wavelength is preferentially absorbed by deoxy-hemoglobin.

38. The method of claim 35, wherein at least one of the first or second wave lengths is generated by a substantially monochromatic light source.

39. The method of claim 35, wherein at least one of the first or second wave lengths is generated by an LED device, a tunable LED device, a laser or a tunable laser.

40. The method of claim 35, further comprising emitting light at a third wavelength onto the skin of the patient.

41. The method of claim 35, wherein the photo acoustic signal is transduced by an, a crystal transducer, a piezo-crystal transducer or an array of transducers.

42. The method of claim 35, wherein the electrical signal is converted into a digital signal before being analyzed.

43. The method of claim 35, wherein the analysis is performed by a processor.

44. The method of claim 35, wherein the analysis is performed by an instruction set executable by the processor.

45. The method of claim 35, wherein the analysis comprises determining a ratio of an amount of oxy-hemoglobin to an amount of deoxy-hemoglobin.

46. The method of claim 35, further comprising:
generating a signal to alert the patient of the presence of a deep vein thrombosis.

47. The method of claim 46, wherein the signal comprises an audio alarm.

48. The method of claim 46, wherein the signal is generated when a threshold level of hemoglobin is detected in the tissue region.

49. The system of claim 48, wherein the threshold level is a minimum level of hemoglobin in an oxygenated state.

50. The system of claim 49, wherein the threshold level of hemoglobin is a ratio of an amount of hemoglobin in a de-oxygenated state to an amount of hemoglobin in an oxygenated state.
